(19) United States
(12) Patent Application Publication
(10) Pub. No.: US 2009/0104257 A1
(43) Pub. Date: Apr. 23, 2009

(54) Liposomal Vancomycin Formulations

(76) Inventors: Xingong Li, Robbinsville, NJ (US); Walter R. Perkins, Pennington, NJ (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/256,692

(22) Filed: Oct. 23, 2008

Related U.S. Application Data
(60) Provisional application No. 60/981,990, filed on Oct. 23, 2007.
(60) Provisional application No. 61/103,725, filed on Oct. 8, 2008.

(51) Int. Cl. A61K 9/127; A61K 38/14

(52) U.S. Cl. 424/450; 514/8

(57) ABSTRACT

The present disclosure relates in part to liposomal vancomycin compositions having low lipid to drug ratios and high concentration of vancomycin. The present disclosure also relates in part to methods of making such compositions.

### RELATED APPLICATIONS

This application claims to the benefit of priority to U.S. Provisional Application Nos. 60/981,990, filed on Oct. 23, 2007, and 61/103,725, filed on Oct. 8, 2008, both of which are herein incorporated by reference.

### BACKGROUND OF THE INVENTION

Vancomycin is a branched tricyclic glycosylated non ribosomal peptide antibiotic produced by the fermentation of the Actinobacteria species Amycolaopsis orientalis. Vancomycin is believed to act by inhibiting proper cell wall synthesis in Gram-positive bacteria. Additionally, vancomycin alters cell membrane permeability and RNA synthesis. Accordingly, vancomycin is generally used in used in the prophylaxis and treatment of infections caused by Gram-positive bacteria that are unresponsive to other types of antibiotics. Vancomycin generally has been used as a treatment of last resort for infections that are resistant to other first line antibiotics. This is because vancomycin is given intravenously for most indications. Additionally, there are toxicity concerns associated with vancomycin, it presents toxicity concerns, and semi-synthetic pencillins have been developed and used preferentially. Nevertheless, the use of vancomycin has increased particularly with the spread of multiple-resistant Staphylococcus aureus (MRSA) beginning in the seventies.

Vancomycin is usually given intravenously because it is unable to cross the intestinal lining. The administration must be slow, using a dilute solution over at least about 60 minutes due to pain and thrombophlebitis. Vancomycin activity is time dependent. Accordingly, its antimicrobial activity depends on the amount of time that the drug level exceeds the minimum inhibitory concentration (MIC) of the target organism. For example, vancomycin is usually administered such that blood levels remain at about 10 to 20 mcg/mL. Intravenous administration of vancomycin in adults is typically about 500 mg by IV infusion for 6 hours or about 1 g for 12 hours. Children receive vancomycin intravenously in an amount of about 10 mg/kg for 6 hours. Infants and newborns may receive vancomycin intravenously in an amount of about 15 mg/kg initially, followed by 10 mg/kg for 12 hours in the first week of life, and every eight hours for ages up to 1 month. Vancomycin is typically administered orally in adults in an amount of about 500 mg to 2 g per day, in 3 or 4 divided doses, for about 7 to 10 days. It is generally administered to children in an amount of about 40 mg/kg/day (up top 2 g/day) in 3 or 4 divided doses for 7 to 10 days.

Vancomycin has been used for the treatment of pseudomembranous colitis, where it is given orally in order to reach the site of infection. Vancomycin has also been used off-label by inhalation using a nebulizer in order to treat respiratory tract infections.

Cystic fibrosis (CF) patients have thick mucous and/or sputum secretions in the lungs, frequent consequential infections, and biofilms resulting from bacterial colonizations. All these fluids and materials create barriers to effectively targeting infections with antiinfectives. One aspect of the present disclosure overcomes these barriers, and even allows reduced dosing (in amount or frequency), thereby reducing the drug load on patients and potentially improving patient compliance. For lung infections generally, the dosing schedule provided by the invention provides a means of reducing drug load.

Cystic fibrosis can also lead to bronchiectasis. Bronchiectasis is an abnormal stretching and enlarging of the respiratory passages caused by mucus blockage. When the body is unable to get rid of mucus, mucus becomes stuck and accumulates in the airways. The blockage and accompanying infection cause inflammation, leading to the weakening and widening of the passages. The weakened passages can become scarred and deformed, allowing more mucus and bacteria to accumulate, resulting in a cycle of infection and blocked airways. Bronchiectasis is a disease that causes localized, irreversible dilatation of part of the bronchial tree. Involved bronchi are dilated, inflamed, and easily collapsible, resulting in airflow obstruction and impaired clearance of secretions. Bronchiectasis is associated with a wide range of disorders, but it usually results from necrotizing bacterial infections, such as infections caused by the Staphylococcus or Klebsiella species or Bordatella pertussis.

Bronchiectasis is one of the chronic obstructive pulmonary diseases (COPD) and it can be complicated by emphysema and bronchitis. The disease is commonly misdiagnosed as asthma or pneumonia. Bronchiectasis can develop at any age, begins most often in childhood, but symptoms may not be apparent until much later. Bronchiectasis can occur as part of a birth defect, such as primary ciliary dyskinesia or cystic fibrosis. About 50% of all cases of bronchiectasis in the U.S. result from cystic fibrosis. It can also develop after birth as a result of injury or other diseases, like tuberculosis, pneumonia and influenza.

Dilation of the bronchial walls results in airflow obstruction and impaired clearance of secretions because the dilated areas interrupt normal air pressure of the bronchial tubes, causing sputum to pool inside the dilated areas instead of being pushed upward. The pooled sputum provides an environment conducive to the growth of infectious pathogens, and these areas of the lungs are thus very vulnerable to infection. The more infections that the lungs experience, the more damaged the lung tissue and alveoli become. When this happens, the bronchial tubes become more inelastic and dilated, which creates a perpetual, destructive cycle within this disease.

There are three types of bronchiectasis, varying by level of severity. Fusiform (cylindrical) bronchiectasis (the most common type) refers to mildly inflamed bronchi that fail to taper distally. In varicose bronchiectasis, the bronchial walls appear beaded, because areas of dilation are mixed with areas of constriction. Saccular (cystic) bronchiectasis is characterized by severe, irreversible ballooning of the bronchi peripherally, with or without air-fluid levels. Chronic productive cough is prominent, occurring in up to 90% of patients with bronchiectasis. Sputum is produced on a daily basis in 76% of patients.

There are both congenital and acquired causes of bronchiectasis. One common genetic cause is Cystic Fibrosis, in which a small number of patients develop severe localized bronchiectasis. Other genetic causes or contributing factors include Kartagener syndrome, Young's syndrome, alpha 1-antitrypsin deficiency, and Primary immunodeficiencies.

Acquired bronchiectasis occurs more frequently, with one of the biggest causes being tuberculosis. A especially common cause of the disease in children is Acquired Immunodeficiency Syndrome, stemming from the human immunodeficiency virus. Other causes of bronchiectasis include respiratory infections, obstructions, inhalation and aspiration of ammonia, and other toxic gases, pulmonary aspiration, alcoholism, heroin use and allergies. Cigarette smoking may also contribute to bronchiectasis.

The diagnosis of bronchiectasis is based on the review of clinical history and characteristic patterns in high-resolution CT scan findings. Such patterns include “tree-in-bud” abnormalities and cysts with definable borders. Bronchiectasis may also be diagnosed without CT scan confirmation if clinical history clearly demonstrates frequent, respiratory infections, as well confirmation of an underlying problem via blood work and sputum culture samples.

Symptoms include coughing (worsened when lying down), shortness of breath, abnormal chest sounds, weakness, weight loss, and fatigue. With infections the mucus may be discolored, foul smelling and may contain blood. Symptom severity varies widely from patient to patient and occasionally, a patient is asymptomatic.

Treatment of bronchiectasis is aimed at controlling infections and bronchial secretions, relieving airway obstruction, and preventing complications. This includes prolonged usage of antibiotics to prevent detrimental infections, as well as eliminating accumulated fluid with postural drainage and chest physiotherapy. Surgery may also be used to treat localized bronchiectasis, removing obstructions that could cause progression of the disease.

Inhaled steroid therapy that is consistently adhered to can reduce sputum production and decrease airway constriction over a period of time will prevent progression of bronchiectasis. One commonly used therapy is beclometasone dipropionate, also used in asthma treatment. Use of inhalers such as Albuterol (Salbutamol), Fluticasone (Flovent/Flixotide) and Ipratropium (Atrovent) may help reduce likelihood of infection by clearing the airways and decreasing inflammation.

Mannitol dry inhalation powder, under the name Bronchitol, has been approved by the FDA for use in Cystic Fibrosis patients with Bronchiectasis. The original orphan drug indication approved in February 2005 allowed its use for the treatment of bronchiectasis. The original approval was based on the results of phase 2 clinical studies showing the product to be safe, well-tolerated, and effective for stimulating mucus hydration/clearance, thereby improving quality of life in patients with chronic obstructive lung diseases like Bronchiectasis. Long-term studies are underway as of 2007 to ensure the safety and effectiveness of the treatment.

Bronchiectasis patients are often given antibiotics for infection and bronchodilator medicines to open passages. Sometimes antibiotics are prescribed for a long period to prevent recurring infections, especially in people who have cystic fibrosis. There are also physical therapy techniques to help clear mucus. Lung transplants are also an option for severe cases. Fatalities are uncommon but may result from massive hemorrhage. If lung infections are treated immediately, bronchiectasis is less likely to develop.

Pneumonia is an illness of the lungs and respiratory system in which the alveoli (microscopic air-filled sacs of the lung responsible for absorbing oxygen from the atmosphere) become inflamed and flooded with fluid. Pneumonia can result from a variety of causes, including infection with bacteria, viruses, fungi, or parasites, and chemical or physical injury to the lungs. Typical symptoms associated with pneumonia include cough, chest pain, fever, and difficulty in breathing. Diagnostic tools include x-rays and examination of the sputum.

Treatment of the above diseases by administering an agent, such as vancomycin, to the lungs of a patient, for example via inhalation, is particularly desirable. Inhalation of a drug delivers the drug more directly to the site of the disease, and minimizes systemic exposure to the drug.

Certain sustained release technology suitable for administration by inhalation employs lipid based formulations, such as liposomes, to provide prolonged therapeutic effect of drug in the lung and systemically by sustained release and the ability to target and enhance the uptake of drug into sites of disease. For a liposomal drug delivery system, it is often desirable to lower the lipid-to-drug (L/D) ratio as much as possible to minimize the lipid load to avoid saturation effects in the body. For lung delivery by inhalation, this may be particularly true because for chronic use, dosing of liposomes could outpace clearance of lipid from the lung, thus limiting the administration and thus effectiveness of the drug product. A lower L/D ratio would allow more drug to be given before the dosing/clearance threshold is met. Additionally, a lower L/D ratio minimizes the amount of time a subject needs to spend undergoing the inhalation treatment since the drug concentration is higher. Thus, a lower L/D ratio can ease administration and increase patient comfort and compliance.

### SUMMARY OF THE INVENTION

It is an object of the present invention to provide lipid based vancomycin formulations with low lipid to drug ratios. In one embodiment, the present invention relates to liposomal vancomycin comprising a liposome and vancomycin. In some embodiments, the vancomycin is encapsulated in the liposome. In other embodiments, the vancomycin is in an aqueous medium encapsulated within a liposome, for example the aqueous medium is an aqueous gel or viscous suspension.

In some embodiments, the vancomycin concentration in the aqueous medium is about 25 to 400, about 25 to 200, about 30 to 175, about 40 to 150, about 40 to 125, about 40 to 100, about 40 to 80, about 45 to 80, about 50 to 75, about 50 to 65, about 40 to 70, about 40 to 60 or about 45 to 55 mg/mL.

In some embodiments, the liposome comprises at least one lipid, and the composition has a lipid to vancomycin ratio of about 3:1 or less. In some embodiments, the lipid to vancomycin ratio is about 0. 1:1 to 3:1. In other embodiments, the lipid to vancomycin ratio is about, about 0.1 to 1.

In some embodiments, the liposome has a mean particle size of about 0.1 to 5, about 0.1 to 2, about 0.1 to 2.5, about 0.5 to 3, about 0.5 to 2, about 1 to 3, about 1.25 to 3 microns, or about 1.5 to 2.5 microns.

In some embodiments, the liposome comprises a lipid selected from the group consisting of phosphatidyl cholines (PCs), phosphatidyl-glycerols (PGs), phosphatidic acids (PAs), phosphatidylinositols (Pls), phosphatidyl serines (PSs), and mixtures thereof. In other embodiments, the liposome comprises a lipid is selected from the group consisting of: egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), tocopherol, ammonium salts of fatty acids, ammonium salts of phospholipids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixtures thereof. In other embodiments, the lipid is a phosphatidyl choline. In other embodiments, the lipid is a saturated phosphatidyl choline, such as dipalmitoylphosphatidylcholine (DPPC).

In some embodiments, the liposome does not comprise a sterol. In other embodiments, the liposome comprises a lipid consisting essentially of a phosphatidyl choline. In other embodiments, the lipid consists essentially of DPPC.

In some embodiments, at least about 50% of the vancomycin remains inside the liposome during a nebulization process.

Another aspect of the invention relates to a method of preparing a vancomycin liposomal formulation comprising:

a) infusing an alcoholic lipid solution into an aqueous/alcoholic vancomycin solution to form an initial vancomycin liposomal formulation; and

b) removing the alcohol to form the vancomycin liposomal formulation. In some embodiments, step b) further comprises removing unencapsulated vancomycin from the vancomycin liposomal formulation.

In some embodiments, the alcohol is ethanol.

In some embodiments, the alcohol is removed by dialysis or diafiltration or centrifugation.

In other embodiments, the aqueous/alcoholic vancomycin solution has a vancomycin concentration of about 100 to 500 mg/mL.

In other embodiments, the alcoholic lipid solution has a lipid concentration of about 50 to 250 mg/mL.

These embodiments of the present invention, other embodiments, and their features and characteristics, will be apparent from the description, drawings and claims that follow.

### BRIEF DESCRIPTION OF THE FIGURES

FIG. 1 depicts a graph showing release of vancomycin from two different liposomal formulations under physiologic conditions.

FIG. 2 depicts leakage of vancomycin from a typical liposomal vancomycin formulation under different storage temperatures.

FIG. 3 depicts a typical liposomal formulation fractionated by a density gradient. The liposomal population was homogeneous and its Lipid/Drug ratio was uniform throughout the population.

FIG. 4 depicts a graph of the survival of Swiss Webster mice with pneumoniae and sepses after treatment with inhaled liposomal and inhaled soluble vancomycin.

FIG. 5 depicts a graph of the survival of Swiss Webster mice with pneumoniae and sepses after treatment with inhaled liposomal and inhaled soluble vancomycin.

FIG. 6 depicts a graph of the Log₁₀CFU/lung in the lungs of mice treated with saline, inhaled liposomal vancomycin and intraperitoneally injected vancomycin.

FIG. 7 depicts a graph of the dose dependent increase of vancomycin levels in the lungs of mice after three days of treatment.

FIG. 8 depicts a graph of the detection of colony forming units (CFU) in the lung after vancomycin exposure under various conditions.

### DETAILED DESCRIPTION OF THE INVENTION

### I. Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The term “pulmonary distress” refers to any disease, ailment, or other unhealthy condition related to the respiratory tract of a human. Generally pulmonary distress results in difficulty of breathing.

The term “treating” is art-recognized and refers to curing as well as ameliorating at least one symptom of any condition or disease.

The term “preventing” is art-recognized and refers to administration to the subject of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The terms “therapeutically effective dose” and “therapeutically effective amount” refer to that amount of a compound that results in prevention or amelioration of symptoms in a patient or a desired biological outcome, e.g., improved clinical signs, delayed onset of disease, reduced levels of bacteria, etc.

A “patient,” “subject” or “host” to be treated by the subject method may mean either a human or non-human animal.

The term “mammal” is known in the art, and exemplary mammals include humans, primates, bovines, porcines, canines, felines, and rodents (e.g., mice and rats).

The term “bioavailable” is art-recognized and refers to a form of the subject invention that allows for it, or a portion of the amount administered, to be absorbed by, incorporated to, or otherwise physiologically available to a subject or patient to whom it is administered.

The term “pharmaceutically-acceptable salts” is art-recognized and refers to the relatively non-toxic, inorganic and organic acid addition salts of compounds, including, for example, those contained in compositions of the present invention.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient.

The term vancomycin refers to a compound of the following formula:

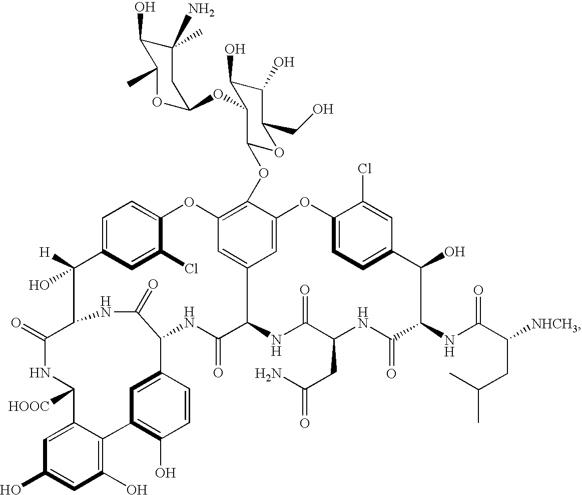

or a pharmaceutically acceptable salt thereof. For example, the salt may be a hydrochloride salt.

In one embodiment, the invention is directed to a liposomal vancomycin composition comprising vancomycin and a liposome, for example, wherein the vancomycin is encapsulated within a liposome. In some embodiments, the vancomycin is in an aqueous medium encapsulated within a liposome. In some embodiments, the aqueous vancomycin inside the liposome has a high vancomycin concentration, thereby forming a viscous suspension or a gel. Thus, the composition comprises an aqueous vancomycin gel or suspension encapsulated by a lipid membrane.

The compositions of the present invention advantageously have a low lipid to vancomycin ratio. For a liposomal drug delivery system, it is often desirable to lower the lipid-to-drug (L/D) ratio as much as possible to minimize the lipid load to avoid saturation effects in the body. In one embodiment, the lipid to vancomycin ratio of the aforementioned compositions is about 3:1 or less, for example, about 0.1:1 to 3:1, about 0.1:1 to 1:1:, about 0.1:1 to 0.9:1, about 0.1:1 to 0.8:1, about 0.2:1 to 0.75:1, about 0.25:1 to 0.7:1, or about 0.35:1 to 0.65:1 by weight. In other embodiments, the L/D ratio is about 0.50, about 0.55, about 0.60, about 0.65 or about 0.70 by weight.

In one embodiment, the aforementioned compositions have a vancomycin concentration in the aqueous medium of about 25 to 200, about 30 to 175, about 40 to 150, about 40 to 125, about 40 to 100, about 40 to 80, about 45 to 80, about 50 to 75, about 50 to 65, about 40 to 70, about 40 to 60, or about 45 to 55 mg/mL. In other embodiments, the vancomycin concentration is about 0.40, about 0.45, about 0.5, about 0.55 or about 0.60 mg/mL.

In another embodiment, the liposome of the aforementioned compositions has a mean particle size of about 0.1 to 5, about 1.0 to 5.0, about 1.0 to 3.0, about 1.0 to 2.0, about 1.25 to 3.0, about 1.5 to 2.5 microns, about 1.0 to 2.0, or about 1.25 to 1.75 microns. In other embodiments, the mean particular size is about 1.0, about 1.1, about 1.2, about 1.3, about 1.4, about 1.5, about 1.6, about 1.7, about 1.8, about 1.9, or about 2.0 microns.

The lipid vancomycin formulations of the present invention may comprise an aqueous dispersion of the liposomes. The formulation may contain lipid excipients to form the liposomes, and salts/buffers to provide the appropriate osmolarity and pH. The formulation may comprise a pharmaceutical excipient. The pharmaceutical excipient may be a liquid, diluent, solvent or encapsulating material, involved in carrying or transporting any subject composition or component thereof from one organ, or portion of the body, to another organ, or portion of the body. Each excipient must be “acceptable” in the sense of being compatible with the subject composition and its components and not injurious to the patient. Suitable excipients include trehalose, raffinose, mannitol, sucrose, leucine, trileucine, and calcium chloride. Examples of other suitable excipients include (1) sugars, such as lactose, and glucose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

### Lipids and Liposomes

The lipids used in the compositions of the present invention can be synthetic, semi-synthetic or naturally-occurring lipids, including phospholipids, tocopherols, steroids, fatty acids, glycoproteins such as albumin, anionic lipids and cationic lipids. The lipids may be anionic, cationic, or neutral. In one embodiment, the lipid formulation is substantially free of anionic lipids, substantially free of cationic lipids, or both. In one embodiment, the lipid formulation comprises only neutral lipids. In another embodiment, the lipid formulation is free of anionic lipids or cationic lipids or both. In another embodiment, the lipid is a phospholipid. Phospholipids include egg phosphatidyl choline (EPC), egg phosphatidylglycerol (EPG), egg phosphatidylinositol (EPI), egg phosphatidylserine (EPS), phosphatidylethanolamine (EPE), and egg phosphatidic acid (EPA); the soya counterparts, soy phosphatidyl choline (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid can be made up of fatty acids of different chain lengths and different degrees of unsaturation. In particular, the compositions of the formulations can include dipalmitoylphosphatidylcholine (DPPC), a major constituent of naturally-occurring lung surfactant as well as dioleoylphosphatidylcholine (DOPC). Other examples include dimyristoylphosphatidylcholine (DMPC) and dimyristoylphosphatidylglycerol (DMPG) dipalmitoylphosphatidcholine (DPPC) and dipalmitoylphosphatidylglycerol (DPPG) distearoylphosphatidylcholine (DSPC) and distearoylphosphatidylglycerol (DSPG), dioleylphosphatidylethanolamine (DOPE) and mixed phospholipids like palmitoylstearoylphosphatidylcholine (PSPC) and palmitoylstearoylphosphatidylglycerol (PSPG), driacylglycerol, diacylglycerol, seranide, sphingosine, sphingomyelin and single acylated phospholipids like mono-oleoyl-phosphatidylethanol amine (MOPE).

The lipids used can include ammonium salts of fatty acids, phospholipids and glycerides, phosphatidylglycerols (PGs), phosphatidic acids (PAs), phosphotidylcholines (PCs), phosphatidylinositols (PIs) and the phosphatidylserines (PSs). The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3- di-(9 (Z)-octadecenyloxy)-prop-1-yl-N,N,N-trimethylammonium chloride (DOTMA) and 1,2-bis(oleoyloxy)-3-(trimethylammonio)propane (DOTAP). Examples of PGs, PAs, PIs, PCs and PSs include DMPG, DPPG, DSPG, DMPA, DPPA, DSPA, DMPI, DPPI, DSPI, DMPS, DPPS and DSPS, DSPC, DPPG, DMPC, DOPC, egg PC.

In another embodiment, the liposome comprises a lipid selected from the group consisting of phosphatidyl cholines (PCs), phosphatidyl-glycerols (PGs), phosphatidic acids (PAs), phosphatidylinositols (Pls), and phosphatidyl serines (PSs).

In another embodiment, the lipid is selected from the group consisting of: egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidyl choline (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), tocopherol, ammonium salts of fatty acids, ammonium salts of phospholipids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixtures thereof.

In another embodiment, the liposome comprises a phosphatidyl choline. The phosphatidyl choline may be unsaturated, such as DOPC or POPC, or unsaturated, such as DPPC. In some embodiments, the phosphatidyl choline is (DPPC). In another embodiment, the liposome does not include a sterol. In one embodiment, the liposome consists essentially of a phosphatidyl choline. In another embodiment, the liposome consists essentially of DPPC.

Liposomes or lipid antiinfective formulations composed of phosphatidylcholines, such as DPPC, aid in the uptake by the cells in the lung such as the alveolar macrophages and helps to sustain release of the antiinfective agent in the lung (Gonzales-Rothi et al. (1991)). The negatively charged lipids such as the PGs, PAs, PSs and PIs, in addition to reducing particle aggregation, can play a role in the sustained release characteristics of the inhalation formulation as well as in the transport of the formulation across the lung (transcytosis) for systemic uptake.

While not being bound by any particular theory, it is believed that when the lipid comprises a neutral lipid, and does not comprise a negatively charged or positively charged phospholipid, the liposomal formulation has improved uptake by the lungs. For example, the liposome my have improved penetration into a biofilm or mucus layer when the lipid comprises only neutral lipids. Exemplary neutral lipids include phosphatidylcholines, such as DPPC.

Liposomes are completely closed lipid bilayer membranes containing an entrapped aqueous volume. Liposomes can be unilamellar vesicles (possessing a single membrane bilayer) or multilamellar vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase. Lipid antiinfective formulations are associations lipid and the antiinfective agent. This association can be covalent, ionic, electrostatic, noncovalent, or steric. These complexes are non-liposomal and are incapable of entrapping additional water soluble solutes. Examples of such complexes include lipid complexes of amphotencin B (Janoff et al., Proc. Nat Acad. Sci., 85:6122 6126, 1988) and cardiolipin complexed with doxorubicin.

A lipid clathrate is a three-dimensional, cage-like structure employing one or more lipids wherein the structure entraps a bioactive agent. Such clathrates are included in the scope of the present invention.

Proliposomes are formulations that can become liposomes or lipid complexes upon corning in contact with an aqueous liquid. Agitation or other mixing may be necessary. Such proliposomes are included in the scope of the present invention.

### Methods of Treatment and Prevention of Pulmonary Disorders

The compositions of the present invention are useful in treating or preventing pulmonary disorders. In particular, the vancomycin compositions of the present invention can be used to treat cystic fibrosis, bronchiectasis, pneumonia, COPD, or pulmonary infections. The pulmonary infection can be a gram positive infection. Among the pulmonary infections that can be treated with the methods of the invention are Pseudomonas (e.g., P. aeruginosa, P. paucimobilis, P. putida, P. fluorescens, and P. acidovorans), staphylococcal, Methicillin-resistant Staphylococcus aureus (MRSA), streptococcal (including by Streptococcus pneumoniae), Escherichia coli, Klebsiella, Enterobacter, Serratia, Haemophilus, Yersinia pesos, Burkholderia pseudomallei, B. cepacia, B. gladioli, B. multivorans, B. vietnamiensis, Mycobacterium tuberculosis, M. avium complex (MAC)(M. avium and M. intracellulare), M. kansasii, M. xenopi, M. marinum, M. ulcerans, or M. fortuitum complex (M. fortuitum and M. chelonei) infections.

In some embodiments, the invention is directed to a method of preventing a pulmonary disorder or infection comprising administering to a subject any one of the aforementioned compositions. In another embodiment, the invention is directed to preventing bronchiectasis. In some embodiments, the administration is pulmonary, for example by intratracheal administration or via an inhalation device. In some embodiments, the administration is via a nebulizer.

Subjects having cystic fibrosis are particularly prone to the aforementioned pulmonary infections. Additionally, the aforementioned pulmonary infections can lead to bronchiectasis, which is not limited to, but often affects cystic fibrosis patients.

To treat infections, the effective amount of the antiinfective will be recognized by clinicians but includes an amount effective to treat, reduce, ameliorate, eliminate or prevent one or more symptoms of the disease sought to be treated or the condition sought to be avoided or treated, or to otherwise produce a clinically recognizable change in the pathology of the disease or condition. Amelioration includes reducing the incidence or severity of infections in animals treated prophylactically. In certain embodiments, the effective amount is one effective to treat or ameliorate after symptoms of lung infection have arisen. In certain other embodiments, the effective amount is one effective to treat or ameliorate the average incidence or severity of infections in animals treated prophylactically (as measured by statistical studies). In some embodiments, the effective amount is sufficient to eradicate the pulmonary infection. By “eradicate” it is meant that the infection can not be detected in the patient using ordinary methods of skill in the art. For example, the infection may be eradicated when CFU in the lung are not detectable.

In one embodiment, at least about 25% of the vancomycin is associated with the liposome after nebulization. In another embodiment, at least about 50% or at least about 60% of the vancomycin is associated with the liposome after nebulization. In another embodiment, about 50 to 95%, about 50 to 80% or about 60 to 75% of the vancomycin is associated with the liposome after nebulization.

In another embodiment, the composition is administered at a vancomycin dose of about 50 to 1000 mg/day, 100 to 500 mg/day, or 250 to 500 mg/day.

In another embodiment, the composition is administered 1 to 4 times a day. In other embodiments, the composition is administered once a day, twice a day, three times a day or four times a day. In other embodiments, the composition may be administered in a daily treatment cycle for a period of time, or may administered in a cycle of every other day, every third day, every fourth day, every firth day, every 6th day or once a week for a period of time, the period of time be from one week to several months, for example, 1, 2, 3, or 4 weeks or 1, 2, 3, 4, 5, or 6 months.

In one embodiment, the pulmonary disorder is cystic fibrosis, bronchiectasis, or a pulmonary infection, such as the aforementioned pulmonary infections.

In some embodiments, the vancomycin is administered in an amount greater than a minimum inhibitory concentration (MIC) for the pulmonary infection. In some embodiments, the MIC of the pulmonary infection is at least about 0.10 micrograms/mL. in other embodiments, the MIC is from about 0.10 microgram/niL to 25 microgram/mL, about 0.10 to 10 micrograms/mL or about 0.10 to 5 micrograms/mL.

In some embodiments, the Log₁₀ CFU in the lung of the subject are reduced. For example, the Log₁₀ CFU can be reduced by at least about 0.5, about 1.0, about 1.5, about 2.0 or about 2.5. In some embodiments, the total CFU in the lung is less than about 1.0, about 0.75, about 0.5, or about 0.25 after administration of the liposomal vancomycin formulation. In other embodiments, the pulmonary infection in the lung of the subject is eradicated. In other embodiments, the pulmonary infection is reduced more than the inhalation treatment of the same dose of free vancomycin. For example, the rate of reduction or eradication of the pulmonary infection in a population of subjects is higher with a treatment with liposomal vancomycin compared to a population treated with the same dose of free inhaled vancomycin. In some embodiments, the reduction across a population treated with inhaled liposomal vancomycin is at least about 20, about 30 , about 40 , about 50, about 70, about 80, or about 90% higher compared to treatment with inhaled free vancomycin. In other embodiments, the pulmonary infection is reduced in a shorter period of time compared to treatment with the same dose of inhaled free vancomycin.

In one embodiment, the present invention allows delivery of the liposomal vancomycin direct to the lungs, thereby reducing or avoiding systemic exposure to the drug. One embodiment of the invention also allows reduced dosing of vancomycin, in amount and/or frequency, thereby reducing drug load on patients. Cystic fibrosis patients have thick mucous and/or sputum secretions in the lungs, frequent consequential infections, and biofilms resulting from bacterial colonizations. Lung infections that are not associated with cystic fibrosis also sometimes are associated with a biofilm or mucus. Such mucus and biofilms create barriers to effectively targeting infections with antibacterial agents.

Liposomal or other lipid delivery systems can be administered for inhalation either as a nebulized spray, powder, or aerosol, or by intratracheal administration. Inhalation administrations are preferred. In some embodiments, the administration is less frequent and/or has an enhanced therapeutic index compared to inhalation of the free drug or a parenteral form of the drug. Additionally, the time for administering the desired therapeutic dose of vancomycin is reduced compared to inhalation of the free drug. Thus, in some embodiments, the liposomal vancomycin formulation is more effective that inhalation of the same amount of the free drug. Liposomes or other lipid formulations are particularly advantageous due to their ability to protect the drug while being compatible with the lung lining or lung surfactant. While not being bound by any particular theory, it is believed that liposomal vancomycin has a depot effect in the lung. As such, the liposomal vancomycin maintains its therapeutic bioavailability for a period of time after administration by inhalation is complete. In some embodiments, this period of time is longer than the amount of time that free vancomycin remains therapeutically available. For example, the therapeutic bioavailabity of the drug maybe longer than 3, 4, 5, 6, 7, 8, 9 or 10 days after treatment, or even longer than two weeks after administration.

In another embodiment, the composition is administered at a vancomycin dose of about 50 to 1000 mg/day, about 100 to 500 mg/day, or about 250 to 500 mg/day. For example, the dose may be about 100 mg, about 200 mg, about 300 mg, about 400 mg, or about 500 mg per day.

### Methods of Preparation

A process for forming liposomes or lipid antiinfective formulations involves a “solvent infusion” process. This is a process that includes dissolving one or more lipids in a small, preferably minimal, amount of a process compatible solvent to form a lipid suspension or solution and then infusing the solution into an aqueous medium containing the vancomycin. Typically a process compatible solvent is one that can be washed away in a aqueous process such as dialysis or diafiltration. Compatible solvents include alcohols, such as ethanol, isopropanol, propanol, and butanol. “Ethanol infusion,” a type of solvent infusion, is a process that includes dissolving one or more lipids in a small, preferably minimal, amount of ethanol to form a lipid solution and then infusing the solution into an aqueous and ethanol medium containing the vancomycin. A “small” amount of solvent is an amount compatible with forming liposomes or lipid complexes in the infusion process.

The methods of the present invention provide an exceptionally high concentration of vancomycin inside the liposome. The resulting liposomal suspensions have a vancomycin concentration of greater than 5 mg/mL. In some embodiments, the liposomal suspension has a vancomycin concentration of greater than 10, 20, 30, 40, 50, 60, 70, 80, 90, or 100 mg/mL, and up to 250 mg/mL. In certain embodiments, the vancomycin concentration of the liposomal formulation ranges from about 40 mg/mL to about 200 mL. In other embodiments, the vancomycin concentration ranges from about 40 to 150 mg/mL, about 50 to 125 mg/mL, or about 50 to 100 mg/mL.

While not being bound to any particular theory, it is believed that the high vancomycin concentration is provided by using a high concentration vancomycin stock solution during the alcohol infusion step of the preparation of liposomal formulations. The high concentration stock solution is achieved by dissolving vancomycin in a mixture of alcohol, e.g. ethanol, and water, instead of using water alone. A higher concentration solution of vancomycin is achieved by a water/alcohol mixture, compared to water alone. Additionally, a high concentration solution of vancomycin in water is very viscous. Again not being bound by any particular theory, it is believed that the high viscosity causes difficulty or impossibility of sterile filtration of the stock solution. Additionally, the viscosity may create problems during the step of infusion of the lipid/ethanol solution in the vancomycin/water solution, yielding less favorable liposome characteristics. Use of a mixture of alcohol and water in the vancomycin stock solution makes sterile filtration of the stock solution possible and provides more favorable liposome characteristics upon infusion with the lipid/alcohol stock solution.

In one embodiment, the invention is directed to a method of preparing a vancomycin liposomal formulation comprising:

a) infusing an alcoholic lipid solution into an aqueous/alcoholic vancomycin solution to form an initial vancomycin liposomal formulation; and

b) removing the alcohol and untrapped vancomycin to form the vancomycin liposomal formulation.

In one embodiment, the alcohol is removed by diafiltration. In another embodiment, the alcohol is removed In another embodiment, the alcohol is ethanol.

In one embodiment, the aqueous/alcoholic vancomycin stock solution has a vancomycin concentration of about 100 to 500, 200 to 400, or 250 to 350 mg/mL.

In one embodiment, the alcohol lipid stock solution has a lipid concentration of about 50 to 250, 50 to 200, or 75 to 125 mg/mL.

The step of infusing the lipid-alcohol solution into the aqueous solution containing the vancomycin can be performed above or below the surface of the aqueous solution containing the vancomycin. Preferably, the step is performed above the surface of the solution.

Liposome or lipid formulation sizing can be accomplished by a number of methods, such as extrusion, sonication and homogenization techniques which are well known, and readily practiced, by ordinarily skilled artisans. Extrusion involves passing liposomes, under pressure, one or more times through filters having defined pore sizes. The filters are generally made of polycarbonate, but the filters may be made of any durable material which does not interact with the liposomes and which is sufficiently strong to allow extrusion under sufficient pressure. Preferred filters include “straight through” filters because they generally can withstand the higher pressure of the preferred extrusion processes of the present invention. “Tortuous path” filters may also be used. Extrusion can also use asymmetric filters, such as Anopore™ filters, which involves extruding liposomes through a branched-pore type aluminum oxide porous filter.

Liposomes or lipid formulations can also be size reduced by sonication, which employs sonic energy to disrupt or shear liposomes, which will spontaneously reform into smaller liposomes. Sonication is conducted by immersing a glass tube containing the liposome suspension into the sonic epicenter produced in a bath-type sonicator. Alternatively, a probe type sonicator may be used in which the sonic energy is generated by vibration of a titanium probe in direct contact with the liposome suspension. Homogenization and milling apparatii, such as the Gifford Wood homogenizer, Polytron™ or Microfluidizer, can also be used to break down larger liposomes or lipid formulations into smaller liposomes or lipid formulations.

The resulting liposomal formulations can be separated into homogeneous populations using methods well known in the art; such as tangential flow filtration. In this procedure, a heterogeneously sized population of liposomes or lipid formulations is passed through tangential flow filters, thereby resulting in a liposome population with an upper and/or lower size limit. When two filters of differing sizes, that is, having different pore diameters, are employed, liposomes smaller than the first pore diameter pass through the filter. This filtrate can the be subject to tangential flow filtration through a second filter, having a smaller pore size than the first filter. The retentate of this filter is a liposomal/complexed population having upper and lower size limits defined by the pore sizes of the first and second filters, respectively.

Lung surfactant allows for the expansion and compression of the lungs during breathing. This is accomplished by coating the lung with a combination of lipid and protein. The lipid is presented as a monolayer with the hydrophobic chains directed outward. The lipid represents 80% of the lung surfactant, the majority of the lipid being phosphatidyl choline, 50% of which is dipalmitoyl phosphatidyl choline (DPPC) (Veldhuizen et al, 1998). The surfactant proteins (SP) that are present function to maintain structure and facilitate both expansion and compression of the lung surfactant as occurs during breathing. Of these, SP-B and SP-C specifically have lytic behavior and can lyse liposomes (Hagwood et al., 1998; Johansson, 1998). This lytic behavior could facilitate the gradual break-up of liposomes. Liposomes can also be directly ingested by macrophages through phagocytosis (Couveur et al., 1991; Gonzales-Roth et al., 1991; Swenson et al, 1991). Uptake of liposomes by alveolar macrophages is another means by which drugs can be delivered to the diseased site.

The lipids preferably used to form either liposomal or lipid formulations for inhalation are common to the endogenous lipids found in the lung surfactant. Liposomes are composed of bilayers that entrap the desired pharmaceutical. These can be configured as multilamellar vesicles of concentric bilayers with the pharmaceutical trapped within either the lipid of the different layers or the aqueous space between the layers. The present invention utilizes unique processes to create unique liposomal or lipid antiinfective formulations. Both the processes and the product of these processes are part of the present invention.

These embodiments of the present invention, other embodiments, and their features and characteristics, will be apparent from the description, drawings and claims that follow.

### EXEMPLIFICATION

### Example 1

### Liposomal Vancomycin Formulations

Liposomal vancomycin formulations were prepared using the methods described above. Specifically, the alcohol used in the lipid stock solution was ethanol. The alcohol used in the aqueous/alcoholic vancomycin stock solution was also ethanol. Formulations were prepared using DPPC, DPPC/CHOL, DOPC/CHOL and POPC/CHOL. The lipid to drug ratios of vancomycin produced using these methods were very low, as shown in Table 1. Concentrations of vancomycin are also shown in Table 1.

TABLE 1
Liposomal vancomycin formulations

| | | Vancomycin |
|---|---|---|
| | Lipid/drug | concentration |
| Lipid Composition | (wt/wt) | (mg/ml) |
| DPPC | 0.29 | 49.64 |
| DPPC | 0.27 | 64.53 |
| DPPC | 0.19 | 58.64 |
| DPPC | 0.63 | 123.02 |
| DPPC | 0.55 | 50.11 |
| DPPC | 0.63 | 43.29 |
| DPPC | 0.41 | 64.28 |
| DPPC | 0.25 | 76.77 |
| DPPC/CHOL (4/1 wt) | 0.85 | 46.85 |
| DPPC/CHOL (2/1 wt) | 2.33 | 8.94 |
| DPPC/CHOL (2/1 wt) | 6.1 | 10.36 |
| DPPC/CHOL (2/1 wt) | 4.63 | 25.54 |
| DOPC/CHOL (4/1 wt) | 4.93 | 12.18 |
| POPC/CHOL (4/1 wt) | 5.48 | 8.81 |

Additional characteristics (mean particle diameter and pH) of selected liposomal vancomycin formulations and the concentrations of the stock solutions used to prepare them are shown in Table 2.

TABLE 2
Characteristics of exemplary Liposomal vancomycin formulations

| | | | Mean | | Vancomycin | |
|---|---|---|---|---|---|---|
| | | | particle | | stock | Lipid stock |
| | | Vancomycin | diameter | | concentration | concentration |
| Lipid | L/D (wt/wt) | (mg/ml) | (micron) | pH | (mg/ml) | (mg/ml) |
| DPPC | 0.55 | 50.11 | 1.9 | 6.15 | 270 | 100 |
| DPPC | 0.41 | 64.28 | 1.7 | 6.08 | 300 | 100 |
| DPPC | 0.63 | 43.29 | 1.7 | 5.9 | 270 | 100 |

### Example 2

### Degradation Study under Biological Conditions

The liposomal formulation of the present invention prevents degradation of vancomycin in a biological environment. Vancomycin is known to degrade to two Crystal Degradation Products (CDP), known as CDP-m and CDP-M. In order to evaluate the stability of the liposomal vancomycin formulations, two formulations (A and B) were diluted into 10% rat serum and incubated at 37° C. and tested for leakage and degradation to CDP using HPLC. An exemplary Formulation A contains vancomycin in a DPPC liposome, as described above. Formulation B contains vancomycin in a DPPC/CHOL liposome.

Both formulations showed less degradation of vancomycin encapsulated in the liposome compared to vancomycin outside of the liposome. (Table 3). Thus, the liposomal formulation liposome appears to reduce the degradation from vancomycin to CDP, especially during the first 4 days of incubation. Formulation A liposome, which contains DPPC, prevented CDP formation more effectively than formulation B, which contains DPPC and cholesterol.

TABLE 3

| | | | total CDP | CDP conversion | CDP conversion |
|---|---|---|---|---|---|
| | Incubation | Leakage | conversion | (%) outside | (%) inside |
| | days | (%) | (%) | liposomes | liposomes |
| Formulation A | 4 | 26.3 | 3.6 | 11.1 | 0.9 |
| | 7 | 54.9 | 14.4 | 20.0 | 7.6 |
| Formulation B | 4 | 5.0 | 5.8 | 15.8 | 5.0 |
| | 7 | 7.1 | 13.9 | 24.6 | 13.1 |

### Example 3

### Drug Release Profile of Formulations A and B

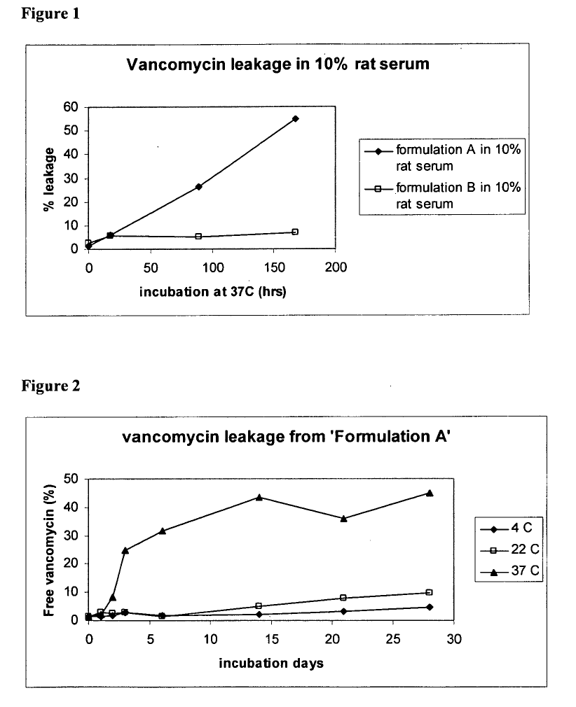

Formulas A and B were incubated in rat serum at in vivo temperature (37° C.). Formulation A shows fast drug release during incubation over a period of 150 hours, while formulation B showed very little release of any drug. (FIG. 1)

### Example 4

### Leakage of Formulation A

A liposomal vancomycin was monitored for its leakage at different storage temperatures. Formulation A was stable at 4° C. The liposomal composition released a substantial amount of vancomycin by increasing the temperature, particularly as the temperature approached the phase transition temperature of DPPC liposome. (FIG. 2). Thus, the liposomal formulations of the present invention should have a long shelf life at temperatures of about 2-8° C., e.g. storage in a refrigerator. The liposomal vancomycin composition is expected to have a good release profile in vivo. Thus, these characteristics are useful for targeted drug release at in vivo temperature.

### Example 5

### Nebulization of Liposomal Vancomycin

A typical liposomal formulation, formulation A, was nebulized by a PARI LC star nebulizer for 20 minutes. The liposomes retained 63% of the vancomycin inside liposome after nebulization.

### Example 6

### Homogeneity of the Liposomal Formulations

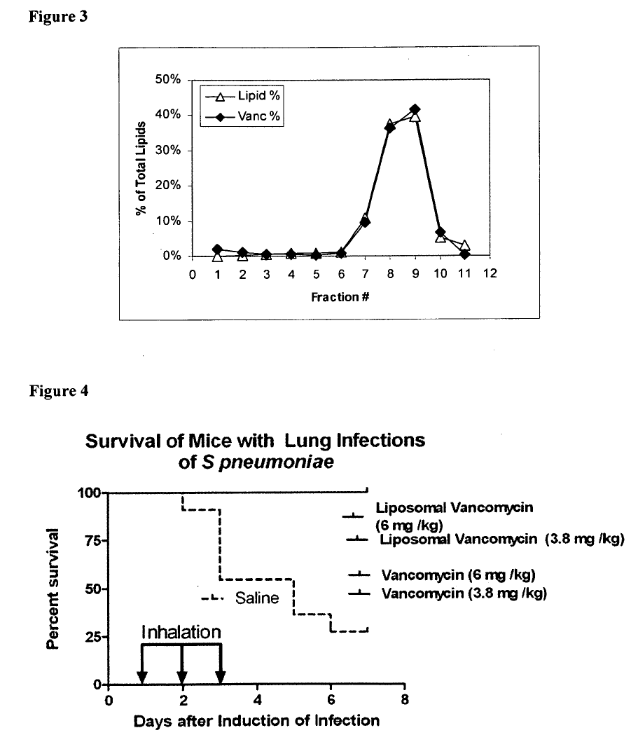

A typical liposomal formulation, formulation A, was fractionated by a density gradient of 0-40% iodiaxanol. The liposomal population was homogeneous and its Lipid/Drug ratio was uniform throughout the population (FIG. 3).

### Example 7

### Fluorescence Anisotropy

Water-soluble fluorescence dye (calcein, 1 mg/ml) was entrapped in two types of liposomal vancomycin. One type contains high concentration of vancomycin and the other contains low concentration of vancomycin. DPPC was added by ethanol injection to make ˜5 mg/ml liposome at 50° C. Free vancomycin and dye was washed by dialysis through 20K MW cutoff tubing against 0.9% saline. Fluorescence anisotropy was measured at an excitation wavelength of 495 nm and emission wavelength of 520 nm. Fluorescence anisotropy is an order parameter, which ranged from 0 to 0.4 in aqueous solution. The higher value indicates more viscous solution.

The anisotropy probed inside the liposome with high concentration of vancomycin was higher (more viscous) than that with low concentration of vancomycin. This suggests that liposomal vancomycin disclosed in this application has high vancomycin concentration (200˜300 mg/ml) inside the liposome, and contains very viscous internal contents.

| | Fluorescence anisotropy |
|---|---|
| Liposomal vancomycin containing low | 0.02 |
| vanc concentration (15 mg/ml) | |
| Liposomal vancomycin containing high | 0.13 |
| vanc concentration (300 mg/ml) | |
| Vancomycin solution (15 mg/ml) | 0.014 |
| control | |
| Vancomycin solution (300 mg/ml) | 0.17 |
| (control) | |

### Example 8

### Comparison of Inhaled Liposomal Vancomycin to Inhaled Soluble Vancomycin in a Murine S. pneumoniae Model

Sixty (60) Thirty six (36) female mice (Swiss Webster, Charles River) were received in the vivarium and acclimated for at least 7 days prior to initiation of the protocol. All mice were instilled via nasal insufflation with S. pneumoniae (ATCC, 6303, 4.1×10⁴ CFU/ mice) after being anesthetized with Ketamine/Xylazine solution (80 mg/kg and 10 mg/kg). The instillation was performed through the nostrils route using micropipette with tips (Gilson, calibrated with Femt Scientific). The mouse is held by its ears and 20 μl of bacteria are gradually (2μl per breath) released into the nostrils (10 μl in each nostril) with the help of a micro-pipette. The mice were observed every 10 min until they fully recovered from the anesthesia.

Mice were dosed on days 1, 2 and 3. There were 5 groups and each group had 12 mice. Mice in group 1 received liposomal vancomycin (6 mg/kg/day, formulation A) by inhalation. Mice in group 2 received liposomal vancomycin (3.8 mg/kg/day, formulation A) by inhalation. Mice in group 3 received free soluble vancomycin (6 mg/kg/day, sterile vancomycin hydrochloride) by inhalation. Mice in group 4 received free soluble vancomycin (3.8 mg/kg/day, sterile vancomycin hydrochloride) by inhalation. Mice in group 5 received sterile normal saline (0.9% NaCl) by inhalation.

Criteria of Euthanization: Surface body temperature of the abdominal region was measured daily using a Raynger MX4 high-performance infrared temperature-scanning thermometer (Raytek, Santa Cruz, Calif.). The mice were held vertical exposing their abdominal region. Their surface body temperatures were taken 3 times. The thermometer averages the 3 reading automatically. The mice were euthanized if the body temperature drops below 28° C. Body weights were measured every day for 7 days and recorded on data sheets (SOP-19). Mice that lost greater than 20% of their initial body weight were euthanized. Mice that didn't meet the above criteria but were moribund were also euthanized at the discretion of the study director.

Determination of bacterial colonies in the lung and blood of mice. Mice that were euthanized prior to day 7 and mice that survived to day 7 were euthanized by CO₂ asphyxiation. The blood was collected by cardiac puncture after euthanization. A 1/10 dilution of blood was made in BHI broth immediately. The lungs were removed aseptically, weighed and placed into 1 ml of BHI broth in a sterile 5 ml Polypropylene round-bottom tube.

Lungs were homogenized sterilely with a PolytronR (Brinkmann, Rexdale, Ontario, Canada) using the maximum speed in the 5 ml Polypropylene round-bottom tube. The tissues were homogenized until a smooth. Ten-fold dilutions of the lung homogenates were made in BHI broth supplemented with 10 ug/ml of Colistin(C) 5 ug/ml of Oxolinic (O). One hundred (100) ul of each dilution of lung homogenates were plated out onto CBO agar plates and spread. The plates were incubated for 24 hours at 37° C. then colonies were counted.

FIG. 4 shows survival of mice infected with the S pneumoniae (ATCC 6303). Survival (100%) of mice that were treated with either formulation of inhaled vancomycin was significantly greater (p<0.0001) than the survival (25%) of mice that received saline by inhalation (FIG. 4). The median survival of the mice in the saline group was day 5. There were 12 mice in each group. Inhalation therapy with saline, liposomal vancomycin and vancomycin occurred on day 1 2 and 3 of the study. Statistics (Log-rank Mantel-Cox test) were performed by Prism® by GraphPad.

The number of bacterial colonies in the lungs and blood of mice that survived to day 7 where determined by the classical agar plate spread method (Table 5). Inhaled soluble vancomycin (6 mg /kg and 3.8 mg /kg) failed to eradicate S. pneumoniae in the lungs in 100 and 92% of the mice respectively (Table 5). These mice had no bacteria in their blood. In contrast 100% of the mice that inhaled liposomal vancomycin (6 mg/kg) had eradicated the bacteria from both the lungs and blood. Furthermore at the lower dose (3.8 mg /kg) of liposomal vancomycin, 58% of the mice had eradicated the bacteria from both the lungs and blood. The 100% of three mice that survived to day 7 in the saline group had bacteria in their lungs and about 66% of these mice had bacteria in their blood (data not shown). These results demonstrate that liposomal vancomycin is more effective in eradicating bacteria from an infective lung than soluble vancomycin.

TABLE 5
Log₁₀ CFU/lung in mice with pneumonia after inhalation
therapy for 3 days

| | Liposomal | Liposomal | Vanco- | | |
|---|---|---|---|---|---|
| | Vancomycin | mycin | mycin | Vancomycin | |
| | (6 mg/kg) | (3.8 mg/kg) | (6 mg/kg) | (3.8 mg/kg) | Saline |
| | Group 1 | Group 2 | Group 3 | Group 4 | Group 5 |
| | Log10 CFU/lung | | | | |
| | *1 | 1 | 4.69 | 1.69 | 2 |
| | 1 | 1 | 2.8 | 1.6 | 1.8 |
| | 1 | 1 | 2.99 | 1 | 1 |
| | 1 | 1 | 2.54 | 1.3 | |
| | 1 | 4.43 | 4.59 | 1.48 | |
| | 1 | 1 | 2.45 | 1.69 | |
| | 1 | 4.45 | 2.39 | 2.23 | |
| | 1 | 1 | 3.52 | 1.84 | |
| | 1 | 1 | 2.92 | 1.84 | |
| | 1 | 3.54 | 2.77 | 1.84 | |
| | 1 | 4.4 | 3.38 | 2 | |
| | 1 | 4.69 | 3.12 | 1.48 | |
| Means | 1 | 2.38 | 3.18 | 1.67 | 1.60 |
| Stdv | 0 | 1.72 | 0.76 | 0.33 | 0.53 |
| t-test | comparator | 0.01 | 1.5E−09 | 4.4E−07 | 0.0006 |
| t-test | 0.01 | comparator | 0.15 | 0.17 | 0.46 |
| t-test | 1.5E−09 | 0.15 | compar- | 2.3E−06 | 0.005 |
| | | | ator | | |
| t-test | 4.4E−07 | 0.46 | 2.3E−06 | comparator | 0.784 |

*Log₁₀ CFU/lung = 1 is the limit of detection; no colonies were detected on the agar plates

Table 6 shows the concentration of vancomycin in the lungs of mice 4 days after the last inhalation therapy. Mice that inhaled liposomal vancomycin had significantly higher concentration of vancomycin in their lung at both doses (6 and 3.8 mg /kg) than mice that inhaled soluble vancomycin (Table 6). S pneumoniae (ATCC 6303) is very sensitive to vancomycin (MIC=0.25 μg /ml). The peak concentration in the lungs/MIC was greater than 200 for both formulations of vancomycin. This increase in the concentration vancomycin in the lungs of mice that inhaled liposomal vancomycin may have resulted in the clearance of the bacteria from the lungs of these mice (Table 5).

TABLE 6
Concentration of vancomycin in the lungs of mice 4
days after inhalation therapy

| | | |
|---|---|---|
| Mouse ID | Vancomycin | Vancomycin |
| # | (μg/lung) | (μg/g) |

Group #1: Liposomal Vancomycin (6 mg/kg)

| | | |
|---|---|---|
| 13 | 11.2 | 81.6 |
| 4 | 10.3 | 66.0 |
| 37 | 11.9 | 92.3 |
| 18 | 8.7 | 55.9 |
| 30 | 10.7 | 63.9 |
| 58 | 22.1 | 170.3 |
| 44 | 13.8 | 83.6 |
| 22 | 18.1 | 106.4 |
| 15 | 12.2 | 81.5 |
| 38 | 11.1 | 69.1 |
| 21 | 21.2 | 137.7 |
| 28 | 14.5 | 104.3 |
| mean | 14 | 93 |
| stdv | 4 | 33 |

Group #2: Liposomal Vancomycin (3.8 mg/kg

| | | |
|---|---|---|
| 27 | 4.9 | 35.1 |
| 51 | 9.0 | 54.9 |
| 11 | 11.8 | 87.1 |
| 34 | 8.1 | 49.1 |
| 46 | 8.5 | 31.7 |
| 25 | 6.9 | 34.1 |
| 59 | 7.3 | 27.4 |
| 53 | 9.0 | 49.6 |
| 0 | 6.8 | 47.7 |
| 12 | 6.7 | 41.5 |
| 49 | 12.9 | 85.8 |
| 24 | 8.3 | 41.9 |
| means | 8 | 49 |
| stdv | 2 | 19 |
| t-test | 0.005 | 0.006 |

Group #3: Soluble Vancomycin (6.0 mg/kg)

| | | |
|---|---|---|
| 45 | 7.6 | 51.6 |
| 19 | 8.8 | 46.1 |
| 20 | 10.4 | 73.9 |
| 14 | 6.4 | 42.0 |
| 54 | 9.7 | 59.0 |
| 47 | 9.0 | 58.5 |
| 36 | 9.3 | 54.9 |
| 6 | 8.3 | 45.8 |
| 41 | 14.0 | 100.5 |
| 56 | 10.3 | 66.0 |
| 50 | 11.2 | 68.3 |
| 2 | 9.8 | 61.8 |
| | 10 | 61 |
| 19 | 2 | 16 |

Group #4: Soluble Vancomycin (3.8 mg/kg)

| | | |
|---|---|---|
| 13 | 5.8 | 45.3 |
| 55 | 5.2 | 37.9 |
| 39 | 6.8 | 52.1 |
| 9 | 3.4 | 27.5 |
| 7 | 4.7 | 26.4 |
| 40 | 4.7 | 21.8 |
| 42 | 5.9 | 29.9 |
| 31 | 2.0 | 14.0 |
| 1 | 3.6 | 21.1 |
| 26 | 5.2 | 40.4 |
| 57 | 8.1 | 58.4 |
| 23 | 6.1 | 37.0 |
| | 5 | 34 |
| | 2 | 13 |
| | 0.0005 | 0.044 |

### Example 9

### Comparison of Inhaled Liposomal Vancomycin to Intraperitoneal Injection of Vancomycin in a Murine S. pneumoniae Model

Thirty six (36) female mice (Swiss Webster, Charles River) were received in the vivarium and acclimated for at least 7 days prior to initiation of the protocol. All mice were instilled via nasal insufflation with S. pneumoniae as described in Example 8. Mice were dosed on days 1, 2 and 3. Mice in Group 1 received 20 min inhalation with liposomal vancomycin (12 mg/kg/day, Formulation A). Mice in Group 2 received intraperitoneal injection of vancomycin (6 mg/kg/day, BID, sterile vancomycin hydrochloride, Lot #NDC0409-6509-010). Mice in Group 3 received 20 min inhalation with sterile 0.9% NaCl for inhalation (Cardinal, Lot #WBA194). Euthanization and determination of bacterial colonies in blood and lungs were performed as described in Example 8.

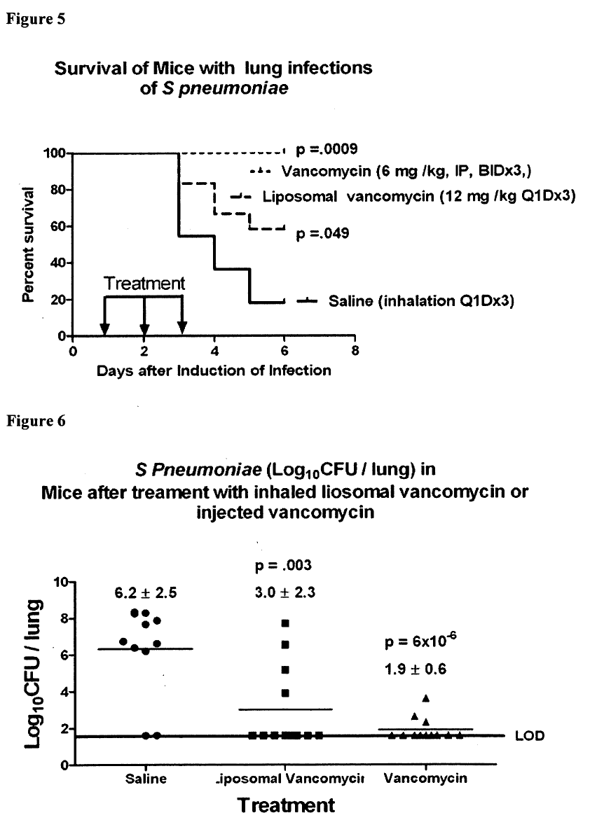

FIG. 5 shows survival of mice infected with the S. pneumoniae (ATCC 6303) and treated with saline, soluble vancomycin, or liposomal vancomycin. Only 16% of the mice that received inhaled saline (n=12) survived to day 7 with the median survival of 4 days. 100% of the mice that received intraperitoneal injections of vancomycin (n=12) survived to day 7, while 58% of the mice that received liposomal vancomycin (n=12) survived to day 7. Survival of mice treated with liposomal and soluble vancomycin were statistically different from the saline treated group (p=0.049 and p=0.0009 respectively). The survival of mice treated with soluble vancomycin was also significantly (p=0.013) different from those that received inhaled liposomal vancomycin. There were 12 mice in each group. Treatment with saline, inhaled liposomal vancomycin and injected (IP) vancomycin occurred on day 1 2 and 3 of the study. Statistics (Log-rank Mantel-Cox test) were performed by Prism® by GraphPad.

The number of bacterial colonies in the lungs and blood of mice that were euthanized and those that survived to day 7 were determined by the classical agar plate spread method (FIG. 6). Even though more mice survived when treated with vancomycin IP, this treatment did not eradicate S. pneumoniae in the lungs in 25% of the mice, and did not eradicate S. pneumoniae from the blood in 42% of the mice. In contrast all the mice that survived after inhalation of liposomal vancomycin had eradicated the bacteria from both the lungs and blood.

Table 7 shows the concentrations of vancomycin in the lungs of mice 4 days after the end of therapy with inhaled liposomal vancomycin or intraperitoneal soluble vancomycin. No detectable concentrations of vancomycin were detected in the lungs of mice that received vancomycin by IP injections. Significant concentrations of vancomycin (44±13 μg/g of lung) were detected in the lungs of mice that received liposomal vancomycin by inhalation (Table 7). The initial mean concentration of vancomycin in the lungs of mice immediately after 20 min of inhalation of liposomal vancomycin was 58±6 μg/g of lung. Although the initial mean concentration of vancomycin in the lungs of mice 30 min after an IP injection of soluble vancomycin (6 mg/kg) was calculated to be 48 μg/g of lung, based on 4.5% deposition of the injected dose in mice with normal lungs, the percentage may have been higher in mice with infected lungs. This result suggests that equal total delivered doses don't equate with equal delivery to the lung since IP injection of soluble vancomycin resulted in higher daily lung dose that did inhaled liposomal vancomycin.

TABLE 7
Concentration of Vancomycin in the lungs of infected mice
4 days post therapy with liposomal or soluble vancomycin

| | | | Treatment: Inhaled Liposomal | | |
|---|---|---|---|---|---|
| Treatment: Soluble | | | Vancomycin | | |
| Vancomycin | | | 12 mg/kg/day) | | |
| (6 mg/kg/day) injected IP | | | | | Vanco- |
| | Vancomycin | Vancomycin | | Vancomycin | mycin |
| mouse | (ug/ | (ug/g of | mouse | (ug/ | (ug/g of |
| ID# | lung) | lung) | ID# | lung) | lung) |
| 12 | #0 | 0 | 13 | 11 | 51 |
| 25 | 0 | 0 | 31 | 6 | 35 |
| 46 | 0 | 0 | 18 | 12 | 67 |
| 41 | 0 | 0 | 34 | 7 | 33 |
| 32 | 0 | 0 | 35 | 9 | 52 |
| 43 | 0 | 0 | 21 | 9 | 30 |
| 47 | 0 | 0 | 27 | 10 | 44 |
| 42 | 0 | 0 | mean | 9 | 44 |
| 44 | 0 | 0 | stdv | 2 | 13 |
| 36 | 0 | 0 | | | |
| 19 | 0 | 0 | | | |
| 26 | 0 | 0 | | | |

#0 = below the level of detection

Mice (n=12) infected with S pneumoniae and treated with 3 daily doses of aerosolized liposomal vancomycin (12 mg/kg) had 58% survival on day 7 of the study. Mice treated with vancomycin (6 mg /kg, BID) by intraperitoneal injections had 100% survival on day 7. In contrast mice (n=12) infected with S pneumoniae and treated with 3 daily doses of aerosolized saline had only 16% of the mice surviving on day 7 with day 4=medium survival. Survival of mice treated with liposomal and soluble vancomycin were statistically different from the saline treated group (p=0.049 and p=0.0009 respectively). The survival of mice treated with soluble vancomycin was also significantly (p=0.013) different from those that received inhaled liposomal vancomycin. Even though more mice survived when treated with vancomycin IP, this treatment did not eradicate S pneumoniae in the lungs in 25% of the mice and did not eradicate S pneumoniae from the blood in 42% of the mice. In contrast all the mice that survived after inhalation of liposomal vancomycin had eradicated the bacteria from both the lungs and blood. Furthermore the initial daily lung dose of liposomal vancomycin and soluble vancomycin were similar (10 ug and 15 ug of vancomycin/lung respectively). The soluble vancomycin concentration was based on a 4% of the delivered dose of vancomycin in the lungs 30 min post IP injections. These results demonstrate that 3 daily doses of aerosolized liposomal vancomycin are very effective in preventing septicemia and in eliminating pneumonia in Swiss Webster mice. But, soluble vancomycin failed to eradicate the infection from the blood and lungs of 42% and 25% of the mice respectively. This beneficial characteristic of liposomal vancomycin may be due to its persistence in the lung after inhalation (6 ug/lung 4 days after the last inhalation therapy) while soluble vancomycin has a very short half-life in the lungs. Six (6) hours after IP injection in Swiss Webster mice no detectable vancomycin was observed.

Table 8 summarizes the above described results, along with the results from a study of 1.2 mg/kg/day of inhaled liposomal vancomycin. The table shows that single daily dose of liposomal vancomycin has even better lung deposition than double dose free vancomycin. (for two dose regimen; 3.8 & 6). For the IP dose, as shown earlier with 6 mg/kg/day, even with 12 mg/kg/day twice a day dose there is no vancomycin deposition in lung was detected after 7 days from completion of treatment.

TABLE 8
Efficacy of liposomal vancomycin in Murine model of S. pneumonia

| | | | # of | [VANCOMYCIN] | | Log |
|---|---|---|---|---|---|---|
| | Route of | | Animals | in | | CFU/mL |
| | Administration | Dose | Surviving | Lung | Log | of |
| Treatment | and Regimen | (mg/kg/day) | to Day 7 | (mg/g)* | CFU/Lung* | Blood* |
| Liposomal | Inhalation; | 1.2 | 7/12 | 29.4 ± 8.3 | 0 | 0 |
| Vancomycin | Q1D × 3 | | | 7/7 | 0/7 | 0/7 |
| Liposomal | Inhalation; | 3.8 | 12/12 | 48.8 ± 18.6 | 1.8 ± 2.1 | ~0** |
| Vancomycin | Q1D × 3 | | | 12/12 | 5/12 | (1/12) |
| Liposomal | Inhalation; | 6.0 | 12/12 | 92.7 ± 31.8 | 0 | 0 |
| Vancomycin | Q1D × 3 | | | 12/12 | 0/12 | 0/12 |
| Free | Inhalation; | 3.8 | 12/12 | 34.31 ± 12.7 | 1.58 ± 0.5 | 0 |
| Vancomycin | BID × 3 | | | 12/12 | 11/12 | 0/12 |
| Free | Inhalation; | 6.0 | 12/12 | 60.7 ± 15.2 | 3.18 ± 0.7 | 0 |
| Vancomycin | BID × 3 | | | 12/12 | 12/12 | 0/12 |
| Free | Intraperitoneal; | 12 | 12/12 | 0 | 0.7 ± 1.3 | 1.7 ± 2.1 |
| Vancomycin | BID × 3 | | | 0/12 | 3/12 | 5/12 |
| Physiological | Inhalation; | NA | 3/12 | NA | 1.6 ± 0.4 | 1.8 ± 1.28 |
| Saline | Q1D × 3 | | | | 3/3 | 2/3 |
| Physiological | Inhalation; | NA | 2/12 | NA | 0 | 0 |
| Saline | Q1\grave{ }D × 3 | | | | 0/2 | 0/2 |

*Data from animals surviving until Day 7 were included in the calculation of average values. Data from animals dying prior to Day 7 were excluded from consideration.
**only one mouse had >1 × 10⁶ bacteria in the blood and >4.69 Log CFUs in the lungs on Day 7. All other mice in this dose group had no CFUs in blood at Day 7

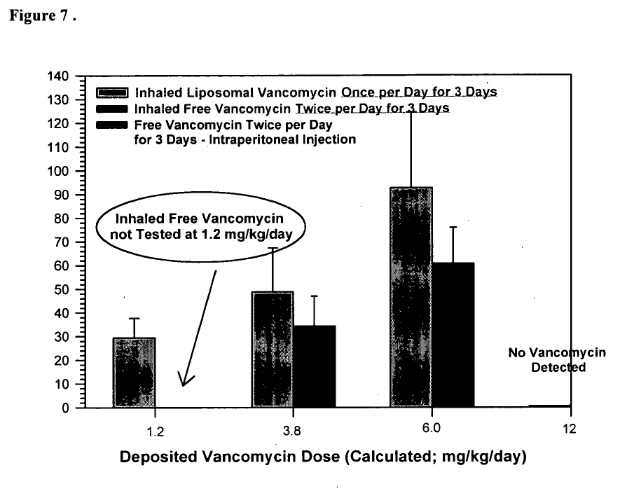

FIG. 7 summarizes the dose dependent increase of vancomycin levels in the lungs of mice at day seven after introduction of S. pneumoniae described in Table 7. Inhaled liposomal vancomycin administered in amounts of 1.2 mg, 3.8 mg and 6 mg/kg/day demonstrated increasing concentration in the lungs at day seven. The concentration was higher compared to inhaled free vancomycin, which was administered at 3.8 and 6.0 mg/kg/day. No vancomycin was detected in the lungs at day seven after IP injection of 12 mg/kg/day of free vancomycin.

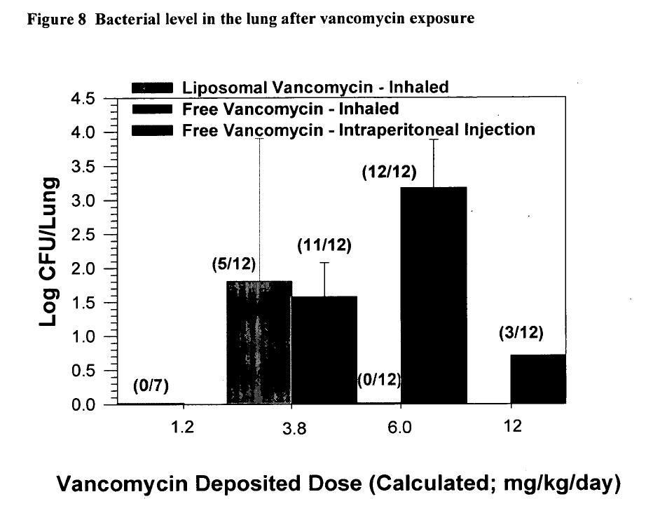

In FIG. 8 the bacterial level in lungs at 7 days after each treatment was estimated in terms of CFU (colony forming unit). For 6.0 mg/kg/day dose liposomal vancomycin completely eradicated the bacteria while all 12 mice treated by same dose of free vancomycin still showed substantial bacterial level. Even in lower dose (3.8 mg/kg/day) bacterial levels are similar for both liposomal and free vancomycin, but only less than 50% of mice showed bacterial level compared to more 90% of mice still infected after free vancomycin treatment.

Incorporation by Reference

All of the U.S. patents and U.S. published patent applications cited herein are hereby incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A liposomal vancomycin comprising a liposome and vancomycin.

2. The composition of claim 1, wherein the vancomycin is encapsulated in the liposome.

3. The composition of claim 2, wherein the vancomycin is in an aqueous medium encapsulated within a liposome.

4. The composition of claim 3, wherein the aqueous medium is an aqueous gel or viscous suspension.

5. The composition of claim 3, wherein the vancomycin concentration in the aqueous medium is about 25 to 400, about 25 to 200, about 30 to 175, about 40 to 150, about 40 to 125, about 40 to 100, about 40 to 80, about 45 to 80, about 50 to 75, about 50 to 65, about 40 to 70, about 40 to 60 or about 45 to 55 mg/mL.

6. The composition of claim 1, wherein the liposome comprises at least one lipid.

7. The composition of claim 6, wherein the composition has a lipid to vancomycin ratio of about 3:1 or less.

8. The composition of claim 7, wherein the lipid to vancomycin ratio is about 0.1:1 to 3:1.

9. The composition of claim 7, wherein the lipid to vancomycin ratio is about, about 0.1 to 1.

10. The composition of claim 1, wherein the liposome has a mean particle size of about 0.1 to 5 microns.

11. The composition of claim 1, wherein liposome comprises a lipid selected from the group consisting of phosphatidyl cholines (PCs), phosphatidyl-glycerols (PGs), phosphatidic acids (PAs), phosphatidylinositols (Pls), phosphatidyl serines (PSs), and mixtures thereof.

12. The composition of claim 1, wherein the liposome comprises a lipid is selected from the group consisting of:
egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidyl choline (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), tocopherol, ammonium salts of fatty acids, ammonium salts of phospholipids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2, 3- di-(9-(Z)-octadecenyloxy)-prop-1-yl-N,N,N-trimethylammonium chloride (DOTMA), 1, 2-bis(oleoyloxy)-3-(trimethylammonio)propane (DOTAP), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixtures thereof.

13. The composition of claim 11, wherein the lipid is a phosphatidyl choline.

14. The composition of claim 13, wherein the lipid is a saturated phosphatidyl choline.

15. The composition of claim 14, wherein the phosphatidyl choline is dipalmitoylphosphatidylcholine (DPPC).

16. The composition of claim 1, wherein the liposome does not comprise a sterol.

17. The composition of claim 1, wherein the liposome comprises a lipid consisting essentially of a phosphatidyl choline.

18. The composition of claim 17, wherein the lipid consists essentially of DPPC.

19. The composition claim 1, wherein at least 50% of the vancomycin remains inside the liposome during a nebulization process.

20. A method of preparing a vancomycin liposomal formulation comprising:
a) infusing an alcoholic lipid solution into an aqueous/alcoholic vancomycin solution to form an initial vancomycin liposomal formulation; and
b) removing the alcohol to form the vancomycin liposomal formulation.

21. The method of claim 20, wherein step b) further comprises removing unencapsulated vancomycin from the vancomycin liposomal formulation.

22. The method of claim 20, wherein the alcohol is ethanol.

23. The method of claim 20, wherein the alcohol is removed by dialysis or diafiltration or centrifugation.

24. The method of claim 20, wherein the aqueous/alcoholic vancomycin solution has a vancomycin concentration of about 100 to 500 mg/mL.

25. The method of claim 20, wherein the alcoholic lipid solution has a lipid concentration of about 50 to 250 mg/mL.
